(19) United States
(12) Patent Application Publication
(10) Pub. No.: US 2024/0115500 A1
(43) Pub. Date: Apr. 11, 2024

(54) POLYMERIC MICELLE COMPLEXES OF MRNA OR ZWITTERIONIC AGENTS, AND FORMULATIONS AND USES THEREOF

(71) Applicant: Aravasc Inc., Sunnyvale, CA (US)

(72) Inventor: Narmada SHENOY, Sunnyvale, CA (US)

(21) Appl. No.: 18/256,620

(22) PCT Filed: Dec. 8, 2021
(86) PCT No.: PCT/US2021/062486

§ 371 Date: Jun. 8, 2023

Related U.S. Application Data
(60) Provisional application No. 63/123,181, filed on Dec. 9, 2020.
(60) Provisional application No. 63/123,145, filed on Dec. 9, 2020.

(51) Int. Cl. A61K 9/107; A61K 31/7125; C08G 81/02

(52) U.S. Cl. CPC A61K 9/1075; A61K 31/7125; C08G 81/025

(57) ABSTRACT

The disclosure provides compositions of polymeric micelle complexes, as well as methods for preparing such compositions. Such compositions are suitable for pharmaceutical delivery of rnRNA or one or more zwitterionic agents to cell interior, and can be used in therapy and/or diagnosis, for example, for treating cancer, inflammation, microbial and viral infections, and metabolic disorders, as well as other diseases.

### CROSS-REFERENCE TO RELATED APPLICATIONS

This application claims priority benefit of U.S. Provisional Application Ser. No. 63/123,181, filed Dec. 9, 2020, and U.S. Provisional Application Ser. No. 63/123,145, filed Dec. 9, 2020, each of which is hereby incorporated by reference in its entirety.

### FIELD

The present invention relates to compositions containing one or more zwitterionic agents, or one or more messenger RNA (mRNA) coding for peptides or proteins, in a polymeric micelle for delivery to the cell interior, and methods of using such compositions in therapy and/or diagnosis, for example, for treating cancer, inflammation, microbial and viral infections, genetic diseases, and metabolic disorders.

### BACKGROUND

It is generally desirable to provide pharmaceutical actives in formulations targeted to the disease site in order to permit lower dosing, reduce dosing frequency, reduce side effects, and/or to improve patient compliance. This may be particularly true in the case of drugs that tend to have unpleasant side effects, low stability and/or undesired degradation, such as certain anti-cancer agents, and anti-viral agents.

Polymer-therapeutics are gaining wide acceptance as drug delivery systems. Polymer-therapeutics involve the use of polymeric systems to enhance the drug's circulation half-life and to reduce its toxicity. Polymeric micelles are formed by spontaneous self-assembly of amphiphilic copolymers. Amphiphilic copolymers are composed of hydrophobic and hydrophilic segments, arranged in either block or graft architecture. Generally speaking, the amphiphilic copolymers in aqueous medium undergo micellization by aggregation of their hydrophobic domains.

Many known polymeric micellar systems are designed to accumulate at the tumor site passively, due to the size of the delivery vehicle, through the leaky vasculature at the tumor site. It is widely recognized that polymeric micellar systems are capable of encapsulating water insoluble agents in the inner hydrophobic core by hydrophobic interactions. However, classical polymeric micelles exhibit poor encapsulation efficiency for water soluble agents. In addition, desire for development of a pharmaceutical preparation which can maintain, if possible, a polymer micelle form under physiological environment over longer period of time shall still be present.

Therefore, there exists a great deal of interest enhancing the loading efficiency and stability in polymeric micellar systems.

### BRIEF SUMMARY

The present disclosure meets the unmet needs described above by providing compositions and kits comprising one or more zwitterionic agents, or one or more messenger RNAs (mRNAs) coding for peptides and/or proteins, complexed with a polymeric micelle. In some embodiments, the zwitterionic agents are peptides and/or proteins. The polymeric micelle complex addresses the loading efficiency issue as well as any potential cytotoxicity and stability problems associated with the zwitterionic agent or mRNA used for therapy, in addition to addressing intracellular delivery.

In some embodiments, the present disclosure provides a composition comprising a polymeric micelle complex comprising:
i) a plurality of block copolymers, wherein each block copolymer comprises at least a pentablock represented by formula (I) of

--[A]-[B]-[C]-[D]-[E]--,
wherein the repeating units of blocks [A] and [E] each independently comprise a pendant moiety carrying a first charge, and
wherein blocks [B]. [C] and [D] are independently poly(alkylene oxide);
wherein the plurality of pentablock copolymers are arranged into a polymeric micelle with an interior hydrophobic core and an exterior hydrophilic layer; and an active agent selected from:
ii) at least one therapeutic agent that is zwitterionic, wherein the therapeutic agent is hydrophobic or hydrophilic in aggregate,
   wherein different hydrophilic and/or hydrophobic regions and/or conformations of the therapeutic agent binds with complementary hydrophobic core and/or exterior hydrophilic layers of the polymeric micelle and/or at least a portion of the pendant moieties in the polymeric micelle to form a polymeric micellar complex; or
iii) mRNA coding for a peptide and/or protein,
   wherein mRNA binds with the pendant moieties in the polymeric micelle based on electrostatic interactions, and binds to the hydrophilic layers of the polymeric micelle to form a stable polymeric micellar complex.

In some embodiments, the present disclosure provides a composition comprising a polymeric micelle complex comprising:
i) a plurality of block copolymers, wherein each block copolymer comprises at least a pentablock represented by formula (I) of --[A]-[B]-[C]-[D]-[E]--,
   wherein the repeating units of blocks [A] and [E] are each independently comprising a pendant moiety carrying a first charge, and
   wherein blocks [B], [C] and [D] are independently poly(alkylene oxide);
   wherein the plurality of pentablock copolymers are arranged into a micelle with an interior hydrophobic core and an exterior hydrophilic layer;
and
ii) at least one therapeutic agent that is zwitterionic, wherein the therapeutic agent is hydrophobic or hydrophilic in aggregate,
   wherein different hydrophobic or hydrophilic regions and/or conformations of the therapeutic agents bind with complementary hydrophobic core and/or exterior hydrophilic layers of the polymeric micelle and/or at least a portion of the pendant moieties in the polymeric micelle to form a polymeric micellar complex.

In some embodiments, the therapeutic agent is a zwitterionic agent. In some embodiments, the zwitterionic agent is a peptide and/or protein. In other embodiments, the therapeutic agent is a peptide and/or protein. In certain embodiments, the peptide and/or protein is SARS-CoV-2 receptor-binding domain (RBD) protein, arginine deiminase, insulin, Glucagon-like Peptide-1 (GLP-1) Receptor Agonists (such as lixisenatide, liraglutide, albiglutide, dulaglutide, semaglutide, exenatide), Lepirudin. Erythropoietin (such as Epoetin alfa, Epoetin zeta). Filgrastim, Glucagon, Interferons (such as Interferon alfa-n3. Interferon beta, Interferon gamma, Natural alpha interferon). Interleukins (such as Interleukin 2 (IL-2), IL-10. IL-12, transforming growth factor-β (TGF-β). IL-27, IL-35 and IL-37), human papilloma virus (HPV) proteins (such as E1, E2, E4, E5, E6 and E7), Desmopressin. or any derivatives and/or metabolites thereof. In some embodiments, the peptide and/or protein is SARS-CoV-2 receptor-binding domain (RBD) protein, lepirudin, insulin, GLP-1 peptides, interferons, or interleukins, or any combinations thereof.

In some embodiments, the present disclosure provides a composition comprising a polymeric micelle complex comprising:
i) a plurality of block copolymers, wherein each block copolymer comprises at least a pentablock represented by formula (I) of --[A]-[B]-[C]-[D]-[E]--,
   wherein the repeating units of blocks [A] and [E] are each independently comprising a pendant moiety carrying a first charge, and
   wherein blocks [B], [C] and [D] are independently poly(alkylene oxide);
   wherein the plurality of pentablock copolymers are arranged into a micelle with an interior hydrophobic core and an exterior hydrophilic layer; and
iii) mRNA coding for a peptide and/or protein, wherein mRNA binds with the pendant moieties in the polymeric micelle based on electrostatic interactions, and binds to the hydrophilic layers of the polymeric micelle to form a stable polymeric micellar complex.

In some embodiments, the mRNA is mRNA coding for SARS-CoV-2 receptor-binding domain (RBD) protein, mRNA coding for erythropoietin, mRNA coding for Glucagon-like-peptide (GLP-1) Receptor Agonists (such as lixisenatide, liraglutide, albiglutide, dulaglutide, semaglutide, exenatide), mRNA coding for Interferons (such as Interferon alfa-n3, Interferon beta, Interferon gamma, Natural alpha interferon), mRNA coding for Interleukins (such as Interleukin 2 (IL-2), IL-10. IL-12, transforming growth factor-β (TGF-β), IL-27, IL-35 and IL-37), mRNA coding for human papilloma virus (HPV) proteins (such as E1, E2, E4, E5, E6 and E7), or a derivative and/or metabolite thereof. In some embodiments, the mRNA codes for SARS-CoV-2 receptor-binding domain (RBD), erythropoietin, interferons, or interleukins, or any combinations thereof.

Other aspects of the present disclosure relate to a pharmaceutical composition containing a composition of any of the embodiments described herein, and a pharmaceutically acceptable carrier.

Other aspects of the present disclosure relate to a kit containing a composition of any of the embodiments described herein for use in any of the methods described herein.

Other aspects of the present disclosure relate to a method for delivering mRNA or one or more zwitterionic agents to a subject in need thereof, by administering to the subject a composition of any of the embodiments described herein.

In one aspect, provided is a method for delivering one or more zwitterionic agents to a subject in need thereof, by administering to the subject a composition of any of the embodiments described herein. Also provided is a method for delivering one or more zwitterionic agents to a cell interior of a subject in need thereof, by administering to the subject a composition of any of the embodiments described herein. In some embodiments, the composition is administered orally, topically, dermally, nasally, intravenously, intramuscularly, intraperitoneally, intracerobrospinally, intracranially, intraspinally, subcutaneously, intraarticularly, intrasynovialy, or intrathecally.

In another aspect, provided is a method for delivering mRNA to a subject in need thereof, by administering to the subject a composition of any of the embodiments described herein. Also provided is a method for delivering mRNA to a cell interior of a subject in need thereof, by administering to the subject a composition of any of the embodiments described herein. In some embodiments, the composition is administered orally, topically, dermally, nasally, intravenously, intramuscularly, intraperitoneally, intracerobrospinally, intracranially, intraspinally, subcutaneously, intraarticularly, intrasynovialy, or intrathecally.

Other aspects of the present disclosure relate to a method for treating cancer, and/or other diseases such as inflammatory diseases, microbial or viral infections, or metabolic disorders, in a subject in need thereof, by administering to the subject a composition of any of the embodiments described herein.

In further aspects, provided are method of preparing a composition of any of the embodiments described herein. In some embodiments, the composition is a polymeric micelle complex. In some embodiments, the composition is a pharmaceutical composition comprising such a polymeric micelle complex.

### DESCRIPTION OF THE DRAWINGS

The present application can be best understood by reference to the following description taken in conjunction with the accompanying figures included in the specification.

FIG. 1 is a gel electrophoresis showing the different results of the various formulations tested in Example 1.

FIG. 2A is a gel electrophoresis showing the results of the semaglutide formulations tested in Example 2.

FIG. 2B is a gel electrophoresis showing the HPV E7 formulations tested in Example 2.

FIG. 2C is a gel electrophoresis showing the interleukin-2 formulations tested in Example 2.

FIG. 2D is a gel electrophoresis showing the interferon alpha and interferon gamma formulations tested in Example 2.

FIG. 3 depicts an agarose gel analysis demonstrating the formation of polymer complexes with mRNA as described in Example 3. Formation of mRNA-PBC complexes was measured by loading of 20 μL of sample at the center of a 1% agarose gel and run for 30 minutes. mRNA in Formulation F samples migrate towards the positive pole (+Ve), while mRNA-PBC in Formulations E, E1, and E2 samples migrate towards the negative pole (−Ve). No signal is detected for Formulation G. The agarose gel was visualized with a LI-COR Odyssey fluorescence gel imager.

### DETAILED DESCRIPTION

The present disclosure is based on the inventors' discovery that certain micelle compositions are effective at complexing with one or more zwitterionic agents and delivering the zwitterionic agent to cell interior.

Unless defined otherwise, all scientific and technical terms are understood to have the same meaning as commonly used in the art to which they pertain. For the purpose of the present disclosure, the following terms are defined.

The term “about” as used herein refers to the usual error range for the respective value readily known to the skilled person in this technical field. Reference to “about” a value or parameter herein includes (and describes) embodiments that are directed to that value or parameter per se. For example. “about x” includes and describes “x” per se. In some embodiments, the term “about” when used in association with a measurement, or used to modify a value, a unit, a constant, or a range of values, refers to variations of +/−2%.

As used herein and in the appended claims, the singular forms “a,” “an,” and “the” include plural reference unless the context clearly indicates otherwise.

### Compositions

Provided herein are compositions comprising a polymeric micelle complex comprising (1) a plurality of block copolymers and (2) at least one therapeutic agent that is zwitterionic.

Polymeric Micelles

A polymeric micelle complex as described herein comprises a plurality of block copolymers. In some embodiments, each block copolymer comprises at least a pentablock represented by formula (I) of --[A]-[B]-[C]-[D]-[E]--, wherein the repeating units of blocks [A] and [E] each independently comprise a pendant moiety carrying a first charge, and wherein blocks [B], [C] and [D] are independently poly(alkylene oxide), so that the plurality of pentablock copolymers are arranged into a micelle with an interior hydrophobic core and an exterior hydrophilic layer.

Block copolymers of the present disclosure can include a hydrophilic and a hydrophobic segment and are able to form polymeric micelles having a core derived from the hydrophobic parts and a shell from the hydrophilic parts. In some embodiments, they exhibit pH-sensitive behavior, good water solubility and capability of thermoreversible gelation.

In some embodiments, block copolymers each block copolymer comprises at least a pentablock represented by formula (I) of --[A]-[B]-[C]-[D]-[E]--, wherein the blocks [A] and [E] each independently have a structure:

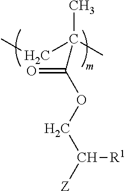

wherein
R¹ is selected from the group consisting of a hydrogen and C₁₋₆ alkyl;
Z is selected from the group consisting of NR²R³. P(OR⁴)₃, SR⁵,

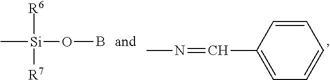

wherein R² and R³ are independently H, C₁₋₆ alkyl, or 1-mer to 28-mer oligonucleotide in which one or more of its natural phosphate backbone linkages are replaced with triazole linkages, or R² and R³ together with the nitrogen form a cyclic amine; R⁴ is C₁₋₆ alkyl; R⁵ is tri(C₁₋₆, alkyl) silyl; and B is C₁₋₆alkyl; and
m is an integer ranging from 1 to 5000.

In some embodiments, the pendant moieties of blocks [A] and [E] are cationic. In some embodiments, the pendant moieties of blocks [A] and [E] are anionic. In some embodiments, the pendant moieties of blocks [A] and [E] are amphiphilic.

In some embodiments, R² and R³ are the same or different C₁₋₆ alkyl, e.g., ethyl. In some embodiments, R² and R³ together with the nitrogen form a cyclic amine, such as pyrrolidine, piperidine, morpholine, and piperazine. In some embodiments, at least one of R² and R³ is 1-mer to 28-mer oligonucleotide in which one or more of its natural phosphate backbone linkages are replaced with triazole linkages.

In some embodiments, the alkylene oxide unit of the blocks FBI and [D] are unsubstituted and unbranched, and the alkylene oxide unit of the block [C] is substituted or branched. In some embodiments, the blocks [B] and [D] are the same

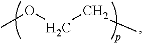

wherein p is an integer ranging from 30 to 20,000. In some embodiments, the blocks [B] and [D] are the same

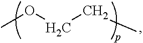

wherein p is about 100.

In some embodiments, the block [C] is

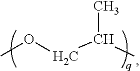

wherein p is an integer ranging from 30 to 20,000. In some embodiments, the block [C] is

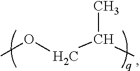

wherein q about 65.

In some embodiments, m is about 13.

In some embodiments, the block copolymers are the ones described in U.S. Pat. No. 7,217,776. In some embodiments, the block copolymers are synthesized by polymerization of a tertiary amine methacrylate with a poly(ethylene oxide)-b-poly (propylene oxide)-b-poly(ethylene oxide) (Pluronic®), including low molecular weight or high molecular weight varieties of said compounds. In certain embodiments, the block copolymers are the one synthesized in Example 2 of U.S. Pat. No. 7,217,776 having the following structure:

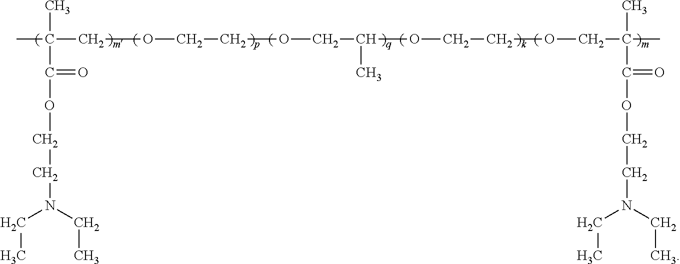

It can be prepared using N,N-(diethyl amino) ethyl methacrylate (DEAEM) as the monomer, disubstituted potassium salt of poly(ethylene oxide)-b-poly(propylene oxide)-b-poly(ethylene oxide) (Pluronic® F127, Mₙ, =12,600, 70% w/w PEG) (Sigma-Aldrich Co St. Louis, Mo.) as the polymerization initiator, and tetrahydrofuran (THF) as the solvent.

In some embodiments, the polymeric micelle complex of the present disclosure have an average particle size that ranges from about 0.01 microns in diameter to about 0.5 microns in diameter. In certain embodiments, the polymeric micelle complex of the present disclosure have an average particle size of about 0.01 microns in diameter, about 0.02 microns in diameter, about 0.03 microns in diameter, about 0.04 microns in diameter, about 0.05 microns in diameter, about 0.06 microns in diameter, about 0.07 microns in diameter, about 0.08 microns in diameter, about 0.09 microns in diameter, about 0.1 microns in diameter, about 0.15 microns in diameter, about 0.2 microns in diameter, about 0.25 microns in diameter, about 0.3 microns in diameter, about 0.35 microns in diameter, about 0.4 microns in diameter, about 0.45 microns in diameter, or about 0.5 microns in diameter. In some embodiments, the polymeric micelle complex of the present disclosure have an average particle size of about 0.025 microns to about 0.25 microns. In some embodiments, the polymeric micelle complex of the present disclosure have an average particle size of about 0.025 microns to about 0.05 microns, about 0.05 microns to about 0.1 microns, about 0.1 microns to about 0.2 microns, about 0.2 microns to about 0.3 microns, about 0.3 microns to about 0.4 microns, or about 0.4 microns to about 0.5 microns.

Therapeutic Agents

Other aspects of the present disclosure relate to compositions containing one or more therapeutic agents complexed with the polymeric micelle of the present disclosure. In some embodiments, the composition comprises a zwitterionic agent. In some embodiments, the composition comprises two, three or four zwitterionic agents. In certain embodiments, the zwitterionic agent is a protein and/or peptide. Any suitable zwitterionic agents known in the art may be used. In some embodiments, the therapeutic agent is a zwitterionic agent. In some embodiments, the zwitterionic agent is a peptide and/or protein. In other embodiments, the therapeutic agent is a peptide and/or protein. In certain embodiments, the peptide and/or protein is SARS-CoV-2 receptor-binding domain (RBD) protein, arginine deiminase, insulin, Glucagon-like Peptide-1 (GLP-1) Receptor Agonists (such as lixisenatide, liraglutide, albiglutide, dulaglutide, semaglutide, exenatide), Lepirudin, Erythropoietin (such as Epoetin alfa. Epoetin zeta), Filgrastim. Glucagon. Interferons (such as Interferon alfa-n3, Interferon beta, Interferon gamma, Natural alpha interferon), Interleukins (such as Interleukin 2 (IL-2), IL-10, IL-12, transforming growth factor-β (TGF-β), IL-27, IL-35 and IL-37), human papilloma virus (HPV) proteins (such as E1, E2, E4, E5, E6 and E7), Desmopressin, or any derivatives and/or metabolites thereof. In some embodiments, the peptide and/or protein is SARS-CoV-2 receptor-binding domain (RBD) protein, lepirudin, insulin, GLP-1 peptides, interferons, or interleukins, or any combinations thereof. By utilizing a temperature and pH responsive polymeric micelle as described herein, one or more zwitterionic agents can be delivered intracellularly.

In some embodiments, two zwitterionic agents are complexed with the polymeric micelle of the present disclosure.

mRNA

Other aspects of the present disclosure relate to compositions containing mRNAs complexed with the polymeric micelle of the present disclosure. In some embodiments, the composition comprises mRNA of a single sequence. In some embodiments, the composition comprises mRNA of two, three or four different sequences.

In some embodiments, the mRNA codes for SARS-CoV-2 receptor-binding domain (RBD) proteins, erythropoietin, (GLP-1) Receptor Agonists (such as lixisenatide, liraglutide, albiglutide, dulaglutide, semaglutide, exenatide), Interferons (such as Interferon alfa-n3, Interferon beta, Interferon gamma, Natural alpha interferon), Interleukines (such as Interleukin 2 (IL-2), IL-10, IL-12, transforming growth factor-β (TGF-β), IL-27, IL-35 and IL-37), human papilloma virus (HPV) proteins (such as E1, E2, E4, E5, E6 and E7), or a derivative and/or metabolite thereof. In some embodiments, the mRNA coding for peptide and/or protein agent is SARS-CoV-2 receptor-binding domain (RBD), erythropoietin, interferons, interleukins in one or more combinations.

By utilizing a temperature and pH responsive polymeric micelle as described herein, one or more mRNAs can be delivered intracellularly.

In some embodiments. mRNAs of two different sequences are complexed with the polymeric micelle of the present disclosure.

Polymeric Micelle Complexes

In one aspect, provided herein is a composition comprising a polymeric micelle complex comprising:
i) a plurality of block copolymers, wherein each block copolymer comprises at least a pentablock represented by formula (I) of

--[A]-[B]-[C]-[D]-[E]--,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer; and an agent selected from:
ii) at least one therapeutic agent that is zwitterionic, wherein the therapeutic agent is hydrophobic or hydrophilic in aggregate.
   wherein different hydrophilic and/or hydrophobic regions and/or conformations of the therapeutic agent binds with complementary hydrophobic core and/or exterior hydrophilic layers of the polymeric micelle and/or at least a portion of the pendant moieties in the polymeric micelle to form a polymeric micellar complex; or
iii) mRNA coding for a peptide and/or protein,
   wherein mRNA binds with the pendant moieties in the polymeric micelle based on electrostatic interactions, and binds to the hydrophilic layers of the polymeric micelle to form a stable polymeric micellar complex.

In one aspect, provided herein is a composition comprising a polymeric micelle complex comprising:
i) a plurality of block copolymers, wherein each block copolymer comprises at least a pentablock represented by formula (I) of --[A]-[B]-[C]-[D]-[E]--,
   wherein the repeating units of blocks [A] and [E] each independently comprise a pendant moiety carrying a first charge, and
   wherein blocks [B], [C] and [D] are independently poly(alkylene oxide);
   wherein the plurality of pentablock copolymers are arranged into a polymeric micelle with an interior hydrophobic core and an exterior hydrophilic layer; and
ii) at least one therapeutic agent that is zwitterionic, wherein the therapeutic agent is hydrophobic or hydrophilic,
   wherein different regions and/or conformations of the therapeutic agent bind with the complementary hydrophobic core and/or exterior hydrophilic layers of the polymeric micelle and/or at least a portion of the pendant moieties in the polymeric micelle to form a polymeric micellar complex.

The one or more zwitterionic agents in the polymeric micelle complex described herein can be present at a molar ratio of zwitterionic agent:block copolymer ranging from about of 0.01 to 99:99:0.01. In some embodiments, the zwitterionic agent is present at a molar ratio of zwitterionic agent:block copolymer ranging from about 0.01:1 to about 1:0.01. In some embodiments, the one or more zwitterionic agents are present at a molar ratio of zwitterionic agent:block copolymer ranging from about 0.01:1 to about 1:1. In some embodiments, the one or more zwitterionic agents are present at a molar ratio of zwitterionic agent:block copolymer of more than or about 0.01:1, more than or about 0.011:1, more than or about 0.0125:1, more than or about 0.015:1, more than or about 0.02:1, more than or about 0.05:1, more than or about 0.1:1, more than or about 0.15:1, more than or about 0.25:1, more than or about 0.3:1, more than or about 0.4:1, more than or about 0.5:1, more than or about 0.6:1, more than or about 0.7:1, more than or about 0.8:1, or more than or about 0.9:1.

As a person with ordinary skill in the art would understand that each block copolymer and zwitterionic agent could carry more than one charges depending on functional groups each has. In some embodiments, the zwitterionic agent in the polymeric micelle complex described herein is present in such an amount that the number of the functional group bearing a first charge in the block copolymer is about 2 times or higher than the number of the functional group bearing the opposite charge in the zwitterionic agent. In some embodiments, the zwitterionic agent is present in such an amount that the molar ratio of the functional group bearing a first charge in the block copolymer/the functional group bearing the opposite charge in the zwitterionic agent ranges from about 1:1 to about 100:1. In some embodiments, the zwitterionic agent is present in such an amount that the molar ratio of the functional group bearing a first charge in the block copolymer/the functional group bearing the opposite charge in the zwitterionic agent is more than or about 2:1, more than or about 3:1, more than or about 4:1, more than or about 5:1, more than or about 10:1, more than or about 20:1, more than or about 30:1, more than or about 40:1, more than or about 50:1, more than or about 60:1, more than or about 70:1, more than or about 80:1, or more than or about 90:1.

In some embodiments, the block copolymer bearing amine groups carries a positive charge and the one or more zwitterionic agents bearing phosphate groups carry a local negative charge.

In some embodiments, the block copolymer bears positively charged amine groups and the one or more zwitterionic agents bear one or more negatively charged phosphate groups, wherein the molar ratio of the positively charged amine groups to the negatively charged phosphate groups (e.g., a molar ratio corresponding to N:P ratios as demonstrated in examples herein) is about 20:1, about 30:1, about 50:1, about 80:1 or about 100:1. In some embodiments, the molar ratio of the positively charged amine groups to the negatively charged phosphate groups ranges from about 10:1 to about 20:1, about 15:1 to about 25:1, about 20:1 to about 30:1, about 25:1 to about 35:1, about 30:1 to about 40:1, about 40:1 to about 50:1, about 45:1 to about 55:1, about 50:1 to about 60:1, about 70:1 to about 80:1, about 75:1 to about 85:1, about 80:1 to about 90:1, about 85:1 to about 95:1, about 90:1 to about 100:1, about 95:1 to about 105:1, or about 100:1 to 110:1. In some embodiments, the molar ratio of the positively charged amine groups to the negatively charged phosphate groups ranges from about 10:1 to about 30:1, about 20:1 to about 40:1, about 40:1 to about 60:1, about 70:1 to about 90:1, or about 90:1 to about 110:1.

In accordance with the present application, the complexation between the block copolymer and one or more zwitterionic agents to form a polymeric micelle complex can be modulated by multiple factors during the complexation process. The complexation process may depend on factors such as molecular structure, size, charge, functional groups, and structural conformation based on the distribution of carboxylic acid, phosphate, thiolate and/or amino groups and composite charge, and regional hydrophobicity or hydrophilcity and conformation. Ionic interactions, alone or in combination with electrostatic interaction, can be modulated by a combination of hydrophobic interactions, hydrogen bonding, structural conformations, pH of the surrounding milieu thus perturbing the formation of stable micellar complexes. To form polymeric micelle complexes in accordance with the present application, the ionic interactions can be modulated by the pKa or the charge on the block copolymer, the pKa or the perturbed pKa values, or the aggregate pKa or isoelectric point (pI) of the zwitterionic agents, structural features, hydrogen bonding and processing condition that allow both the block copolymer and the zwitterionic agents to ionize. In some embodiments, the zwitterionic agent bears one or more carboxylic acid, phosphate, thiolate and amino groups, the resulting conformation, composite charge and isoelectric point (pI) of which are appropriate for complexation.

Polymeric micelle complexes can further comprise one or more secondary agents. The use of a secondary agent in preparations of polymeric micelle complex with one or more zwitterionic agents, such as one or more small molecule drugs can increase the efficiency of said drugs.

In one aspect, provided herein is a composition comprising a polymeric micelle complex comprising:
i) a plurality of block copolymers, wherein each block copolymer comprises at least a pentablock represented by formula (I) of --[A]-[B]-[C]-[D]-[E]--,
   wherein the repeating units of blocks [A] and [E] each independently comprise a pendant moiety carrying a first charge, and
   wherein blocks [B], [C] and [D] are independently poly(alkylene oxide);
   wherein the plurality of pentablock copolymers are arranged into a polymeric micelle with an interior hydrophobic core and an exterior hydrophilic layer; and
iii) mRNA coding for a peptide and/or protein, wherein the mRNA binds with the pendant moieties in the polymeric micelle based on electrostatic interactions, and the different regional hydrophilic and/or the hydrophobic regions of the therapeutic agent binds with the complementary hydrophobic core and/or exterior hydrophilic layers of the polymeric micelle to form a stable polymeric micellar complex.

The mRNA in the polymeric micelle complex described herein can be present at a molar ratio of mRNA:block copolymer ranging from about of 0.01 to 99:99:0.01. In some embodiments, the mRNA is present at a molar ratio of mRNA:block copolymer ranging from about 0.01:1 to about 1:0.01. In some embodiments, the one or more mRNAs are present at a molar ratio of mRNA:block copolymer ranging from about 0.01:1 to about 1:1. In some embodiments, the mRNA are present at a molar ratio of mRNA:block copolymer of more than or about 0.01:1, more than or about 0.011:1, more than or about 0.0125:1, more than or about 0.015:1, more than or about 0.02:1, more than or about 0.05:1, more than or about 0.1:1, more than or about 0.15:1, more than or about 0.25:1, more than or about 0.3:1, more than or about 0.4:1, more than or about 0.5:1, more than or about 0.6:1, more than or about 0.7:1, more than or about 0.8:1, or more than or about 0.9:1.

As a person with ordinary skill in the art would understand that each block copolymer and mRNA could carry more than one charges depending on functional groups each has. In some embodiments, the mRNA in the polymeric micelle complex described herein is present in such an amount that the number of the functional group bearing a first charge in the block copolymer is about 2 times or higher than the number of the functional group bearing the opposite charge in the mRNA. In some embodiments, the mRNA is present in such an amount that the molar ratio of the functional group bearing a first charge in the block copolymer/the functional group bearing the opposite charge in the mRNA ranges from about 1:1 to about 100:1. In some embodiments, the mRNA is present in such an amount that the molar ratio of the functional group bearing a first charge in the block copolymer/the functional group bearing the opposite charge in the mRNA is more than or about 2:1, more than or about 3:1, more than or about 4:1, more than or about 5:1, more than or about 10:1, more than or about 20:1, more than or about 30:1, more than or about 40:1, more than or about 50:1, more than or about 60:1, more than or about 70:1, more than or about 80:1, or more than or about 90:1.

In some embodiments, the block copolymer carries a positive charge and the one or more mRNAs carry a negative charge. In some embodiments, the block copolymer bearing amine groups carries a positive charge and the one or more mRNAs bearing phosphate groups carry a negative charge.

In some embodiments, the block copolymer bears positively charged amine groups, wherein the molar ratio of the positively charged amine groups to the negatively charged phosphate groups of the mRNA phosphate backbone (e.g., a molar ratio corresponding to N:P ratios as demonstrated in examples herein) is about 20:1, about 30:1, about 50:1, about 80:1 or about 100:1. In some embodiments, the molar ratio of the positively charged amine groups to the negatively charged phosphate groups ranges from about 10:1 to about 20:1, about 15:1 to about 25:1, about 20:1 to about 30:1, about 25:1 to about 35:1, about 30:1 to about 40:1, about 40:1 to about 50:1, about 45:1 to about 55:1, about 50:1 to about 60:1, about 70:1 to about 80:1, about 75:1 to about 85:1, about 80:1 to about 90:1, about 85:1 to about 95:1, about 90:1 to about 100:1, about 95:1 to about 105:1, or about 100:1 to 110:1. In some embodiments, the molar ratio of the positively charged amine groups to the negatively charged phosphate groups ranges from about 10:1 to about 30:1, about 20:1 to about 40:1, about 40:1 to about 60:1, about 70:1 to about 90:1, or about 90:1 to about 110:1.

In some embodiments, the molar ratio of the positively charged amine groups to the negatively charged phosphate groups of the mRNA phosphate backbone is about 30:1, or ranges from about 25:1 to about 35:1 or about 20:1 to about 40:1.

In accordance with the present application, the complexation between the block copolymer and mRNA to form a polymeric micelle complex can be modulated by multiple factors during the complexation process. The complexation process may depend on factors such as molecular structure, size, charge, functional groups, and structural conformation. Ionic interactions, alone or in combination with electrostatic interaction, can be modulated by a combination of hydrophobic interactions, hydrogen bonding, structural conformations, pH of the surrounding milieu thus perturbing the formation of stable micellar complexes. To form polymeric micelle complexes in accordance with the present application, the ionic interactions can be modulated by the pKa or the charge on the block copolymer, the pKa or the perturbed pKa values of the mRNA, structural features, hydrogen bonding and processing condition that allow both the block copolymer and the mRNAs to ionize. In some embodiments, the phosphate groups of the mRNA phosphate backbone have a pKa appropriate for complexation.

Polymeric micelle complexes can further comprise one or more secondary agents. The use of a secondary agent in preparations of polymeric micelle complex with mRNA, such as one or more small molecule drugs can increase the efficiency of said drugs.

Secondary Agents

In some embodiments, the secondary agent is a therapeutic or diagnostic agent, such as a dye (e.g., an imaging dye) and a nucleic acid (e.g., DNA and RNA). For example, indocyanine green (ICG) can be used as an imaging dye in the polymeric micelle complex. In some embodiments, the secondary agent is a hydrophobic agent with a solubility less than 10 μg/ml. In some embodiments, the secondary agent is a hydrophobic agent with a solubility greater than 10 ng/ml. In some embodiments, the secondary agent is complexed to the polymeric micelle via hydrophobic interaction.

In some embodiments, the secondary agent is present in the polymeric micelle complex in the amount of up to about 0.5 weight percent, about 1 weight percent, about 5 weight percent, about 10 weight percent, or about 20 weight percent.

### Pharmaceutical Compositions

Compositions of the present disclosure, such as a polymeric micelle complex containing block copolymers complexed with one or more mRNAs or one or more zwitterionic agents, can be incorporated into a variety of formulations for therapeutic use (e.g., by administration) or in the manufacture of a medicament (e.g., for delivering one or more mRNAs or one or more zwitterionic agents of the present disclosure to a subject in need thereof and/or cell interior of a subject in need thereof and/or for treating or preventing a disease or disorder such as cancer, inflammatory disease, microbial infection, viral infection, or metabolic disorder in a subject in need thereof) by combining the composition with appropriate carriers (including, for example, pharmaceutically acceptable carriers or diluents), and may be formulated, for example, into preparations in liquid, aerosolized, semisolid, or powder forms.

In some embodiments, carriers include pharmaceutically acceptable carriers, excipients, or stabilizers that are nontoxic to the cell or subject being exposed thereto at the dosages and concentrations employed. Often the physiologically acceptable carrier is an aqueous pH buffered solution. Suitable physiologically acceptable carriers include, for example, buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. In some embodiments, the polymeric micelle complex described herein is formulated in a buffer in the pH range of 4.5-8.0.5.0-8.0, or 5.5-7.5.

Suitable formulations include, for example, solutions, injections, inhalants, microspheres, aerosols, gels, ointments, creams, lotions, powders, dry vesicular powders, tablets, and capsules. Pharmaceutical compositions can include, depending on the formulation desired, pharmaceutically-acceptable, non-toxic carriers of diluents, which are vehicles commonly used to formulate pharmaceutical compositions for animal or human administration. The diluent is selected so as not to affect the biological activity of the combination. Such diluents include, for example, distilled water, buffered water, physiological saline, PBS, Ringer's solution, dextrose solution, and Hank's solution. A pharmaceutical composition or formulation of the present disclosure can further include, for example, other carriers or non-toxic, nontherapeutic, nonimmunogenic stabilizers, and excipients. The compositions can also include additional substances to approximate physiological conditions, such as pH adjusting and buffering agents, toxicity adjusting agents, wetting agents and detergents. A pharmaceutical composition of the present disclosure can also include any of a variety of stabilizing agents, such as an antioxidant for example.

For oral administration, the active ingredient can be administered in solid dosage forms, such as capsules, tablets, and powders, or in liquid dosage forms, such as elixirs, syrups, and suspensions. The active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate. Examples of additional inactive ingredients that may be added to provide desirable color, taste, stability, buffering capacity, dispersion or other known desirable features are red iron oxide, silica gel, sodium lauryl sulfate, titanium dioxide, and edible white ink. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for parenteral administration (e.g. intrathecal, intramuscular (IM), subcutaneous (SC) and intravenous (IV)), include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In some embodiments, the polymeric micelle complex described herein are formulated for parenteral administration.

Pharmaceutical compositions of the present disclosure containing a composition containing a polymeric micelle complex of the present disclosure may be used (e.g., administered to a subject in need of treatment with a carbohydrate of the present disclosure, such as a human individual) in accord with known methods, such as oral administration, intravenous administration as a bolus or by continuous infusion over a period of time, by intramuscular, intraperitoneal, intracerobrospinal, intracranial, intraspinal, subcutaneous, infra-articular, intrasynovial, intrathecal, oral, topical, or inhalation routes. In some embodiments, compositions and formulations of the present disclosure are useful for subcutaneous (SC), intravenous (IV) or intrathecal administration.

Dosages and desired concentration of pharmaceutical compositions of the present disclosure may vary depending on the particular use envisioned. The determination of the appropriate dosage or route of administration is well within the skill of an ordinary artisan. Animal experiments provide reliable guidance for the determination of effective doses for human therapy, interspecies scaling of effective doses can be performed following the principles described in Mordenti, J. and Chappell. W. “The Use of Interspecies Scaling in Toxicokinetics,” In Toxicokinetics and New Drug Development, Yacobi et al., Eds, Pergamon Press, New York 1989, pp. 42-46.

For in vivo administration of any of the compositions of the present disclosure containing a polymeric micelle complex, normal dosage amounts may vary from 10 ng/kg up to 100 mg/kg of a subject's body weight per day.

Administration of a composition of the present disclosure containing a polymeric micelle complex can be continuous or intermittent, depending, for example, on the recipient's physiological condition, whether the purpose of the administration is therapeutic or prophylactic, and other factors known to skilled practitioners.

It is within the scope of the present disclosure that different formulations will be effective for different treatments and different disorders, and that administration intended to treat a specific organ or tissue may necessitate delivery in a manner different from that to another organ or tissue. Moreover, dosages may be administered by one or more separate administrations, or by continuous infusion. For repeated administrations over several days or longer, depending on the condition, the treatment is sustained until a desired suppression of disease symptoms occurs. However, other dosage regimens may be useful. The progress of this therapy is easily monitored by conventional techniques and assays.

Thus, in some variations, the compositions provided herein may be chronically or intermittently administered to a subject (including, for example, a human) in need thereof. In certain variations, chronic administration is administration of the medicament(s) in a continuous as opposed to acute mode, so as to maintain the initial therapeutic effect (activity) for an extended period of time. In certain variations, intermittent administration is treatment that is not consecutively done without interruption, but rather is cyclic in nature.

### Therapeutic Uses

The present disclosure provides compositions containing a polymeric micelle complex that are capable of delivering one or more mRNAs or one or more zwitterionic agents into the interior of a cell. These compositions are useful for delivering any mRNA or zwitterionic agent of the present disclosure to a subject in need of such agent.

In some embodiments, the subject is a mammal, such as a human, domestic animal, such as a feline or canine subject, farm animal (e.g., bovine, equine, caprine, ovine, and porcine subject), wild animal (whether in the wild or in a zoological garden), research animal, such as mouse, rat, rabbit, goat, sheep, pig, dog, and cat, and birds. In one embodiment, the subject is a human.

In some embodiments, a subject of this disclosure may have any type of cancer, infectious diseases and/or metabolic disorders. In some variations, the subject has cancer. In some variations, the subject has inflammation, microbial infections or viral infections.

Examples of cancer can include, but are not limited to, adrenal cancer, anal cancer, bile duct cancer, bladder cancer, cancer of the blood, bone cancer, a brain tumor, breast cancer, cancer of the cardiovascular system, cervical cancer, colon cancer, cancer of the digestive system, cancer of the endocrine system, endometrial cancer, esophageal cancer, eye cancer, gallbladder cancer, a gastrointestinal tumor, kidney cancer, laryngeal cancer, leukemia, liver cancer, lung cancer, lymphoma, mesothelioma, cancer of the muscular system, myelodysplastic syndrome, myeloma, nasal cavity cancer, nasopharyngeal cancer, cancer of the nervous system, cancer of the lymphatic system, oral cancer, oropharyngeal cancer, ovarian cancer, pancreatic cancer, penile cancer, pituitary tumors, prostate cancer, cancer of the reproductive system, cancer of the respiratory system, a sarcoma, salivary gland cancer, skeletal system cancer, skin cancer, small intestine cancer, stomach cancer, testicular cancer, thymus cancer, thyroid cancer, bladder cancer, or vaginal cancer. The term ‘lymphoma’ may refer to any type of lymphoma including B-cell lymphoma (e.g., diffuse large B-cell lymphoma, follicular lymphoma, small lymphocytic lymphoma, mantle cell lymphoma, marginal zone B-cell lymphoma. Burkitt lymphoma, lymphoplasmacytic lymphoma, hairy cell leukemia, or primary central nervous system lymphoma) or a T-cell lymphoma (e.g., precursor T-lymphoblastic lymphoma, or peripheral T-cell lymphoma). In some embodiments, compositions and formulations containing a polymeric micelle complex as described herein are useful in treating colorectal cancer (CRC), gastrointestinal (GI) cancers, breast cancer, prostate cancer, and head & neck cancer. In some embodiments, compositions and formulations containing a polymeric micelle complex as described herein are useful in treating CRC.

Examples of cancer include cancers that cause solid tumors as well as cancers that do not cause solid tumors. Furthermore, any of the cancers mentioned herein may be a primary cancer (e.g., a cancer that is named after the part of the body where it first started to grow) or a secondary or metastatic cancer (e.g., a cancer that has originated from another part of the body).

In some embodiments, compositions and formulations containing a polymeric micelle complex as described herein are useful in treating a microbial or viral infection, for example, SARS-CoV-2.

In some embodiments, compositions and formulations containing a polymeric micelle complex as described herein are useful in treating microbial infections, viral infections, genetic diseases, and/or metabolic disorders. Suitable mRNAs that may be used include, but are not limited to, mRNA coding for SARS-CoV-2 receptor-binding domain (RBD) protein, mRNA coding for erythropoietin, mRNA coding for (GLP-1) Receptor Agonists (such as lixisenatide, liraglutide, albiglutide, dulaglutide, semaglutide, exenatide), mRNA coding for Interferons (such as Interferon alfa-n3. Interferon beta, interferon gamma, Natural alpha interferon), mRNA coding for Interleukines (such as interleukin 2 (IL-2), IL-10. IL-12, transforming growth factor-β (TGF-β), IL-27, IL-35 and IL-37), mRNA coding for human papilloma virus (HPV) proteins (such as E1, E2. E4, E5, E6 and E7), or a derivative and/or metabolite thereof. In some embodiments, the mRNA coding for peptide and/or protein agent is SARS-CoV-2 receptor-binding domain (RBD), erythropoietin, interferons, or interleukins, or any combinations thereof.

In some embodiments, “treatment” or “treating” includes an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

In some embodiments. “prevention” or “preventing” includes any treatment of a disease or condition that causes the clinical symptoms of the disease or condition not to develop. Compounds may, in some embodiments, be administered to a subject (including a human) who is at risk or has a family history of the disease or condition.

In some variations, an “effective amount” is at least an amount effective, at dosages and for periods of time necessary, to achieve the desired therapeutic or prophylactic result. An effective amount can be provided in one or more administrations.

In some variations, a “therapeutically effective amount” is at least the minimum concentration required to effect a measurable improvement of a particular disease, disorder, or condition, such as a congenital disorder of glycosylation. A therapeutically effective amount herein may vary according to factors such as the disease state, age, sex, and weight of the subject, and the ability of the lipid compositions of the present disclosure to elicit a desired response in the subject. A therapeutically effective amount is also one in which any toxic or detrimental effects of the lipid compositions of the present disclosure are outweighed by the therapeutically beneficial effects.

In one aspect, provided herein is a method for delivering one or more mRNAs or one or more zwitterionic agents to a subject in need thereof. In some embodiments, the method comprises administering to the subject any of the compositions described herein.

In another aspect, provided herein is a method for delivering one or more mRNAs or one or more zwitterionic agents to a cell interior of a subject in need thereof. In some embodiments, the method comprises administering to the subject any of the compositions described herein. In some embodiments, at least a portion of the administered composition traverses the cell plasma membrane to deliver the mRNA or zwitterionic agent to the cell interior.

In another aspect, provided herein is a method for treating cancer and/or other diseases such as liver diseases, in a subject in need thereof. In some embodiments, the method comprises administering to the subject any of the compositions described herein.

In another aspect, provided herein is a method for treating cancer and/or other diseases such as microbial infections, viral infections, genetic diseases, and metabolic disorders, in a subject in need thereof. In some embodiments, the method comprises administering to the subject any of the compositions described herein.

### Articles of Manufacture and Kits

The present disclosure also provides articles of manufacture and/or kits containing a composition of the present disclosure containing a polymeric micelle complex. Articles of manufacture and/or kits of the present disclosure may include one or more containers comprising a purified composition of the present disclosure. Suitable containers may include, for example, bottles, vials, syringes, and IV solution bags. The containers may be formed from a variety of materials such as glass or plastic. In some embodiments, the articles of manufacture and/or kits further include instructions for use in accordance with any of the methods of the present disclosure. In some embodiments, these instructions comprise a description of administration of the composition containing a polymeric micelle complex to deliver the carbohydrate to a subject in need thereof, to deliver one or more mRNAs or one or more zwitterionic agents to a cell interior of a subject in need thereof, or to treat cancer to a subject in need thereof, according to any of the methods of the present disclosure.

The instructions generally include information as to dosage, dosing schedule, and route of administration for the intended treatment. The containers may be unit doses, bulk packages (e.g., multi-dose packages) or sub-unit doses. Instructions supplied in the articles of manufacture and/or kits of the present disclosure are typically written instructions on a label or package insert (e.g., a paper sheet included in the article of manufacture and/or kit), but machine-readable instructions (e.g., instructions carried on a magnetic or optical storage disk) are also acceptable.

The label or package insert indicates that the composition is used for delivering one or more zwitterionic agents and/or treating cancer, inflammatory disease, microbial infections, viral infections, or metabolic disorders. Alternatively, the label or package insert indicates that the composition is used for delivering mRNA and/or treating cancer, microbial infections, viral infections, genetic diseases, or metabolic disorders. Instructions may be provided for practicing any of the methods described herein.

The articles of manufacture and/or kits of the present disclosure may be in suitable packaging. Suitable packaging includes, for example, vials, bottles, jars, and flexible packaging (e.g., sealed Mylar or plastic bags). Also contemplated are packages for use in combination with a specific device, such as an inhaler, nasal administration device (e.g., an atomizer) or an infusion device such as a minipump. An article of manufacture and/or kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). At least one active agent in the composition is one or more zwitterionic agents capable of treating cancer, inflammatory disease, microbial infections, viral infections, or metabolic disorders, and/or improving one or more symptoms thereof. Alternatively, at least one active agent in the composition is mRNA capable of treating cancer, microbial infections, viral infections, genetic diseases, or metabolic disorders, and/or improving one or more symptoms thereof. The container may further comprise a second active agent.

Articles of manufacture and/or kits may optionally provide additional components such as buffers and interpretive information. Normally, the article of manufacture and/or kit comprises a container and a label or package insert(s) on or associated with the container.

### Examples

Articles of manufacture and/or kits may optionally provide additional components such as buffers and interpretive information. Normally, the article of manufacture and/or kit comprises a container and a label or package insert(s) on or associated with the container.

The following Examples are merely illustrative and is not meant to limit any aspects of the present disclosure in any way.

In the following Examples, the pentablock copolymer (“PBC”) micelles are made of a Pluronic F127 (poly(ethyleneoxide)-block-poly(propyleneoxide)-block poly(ethyleneoxide) (PEO-PPO-PEO) and pH-responsive cationic (poly(2-diethylaminoethyl methacrylate) (PDEAEM) as the end blocks. The amphiphilic Pluronic F127 blocks enhance cellular uptake, while the protonatable tertiary amine groups of PDEAEM facilitate endosomal escape and efficient payload release in the cytoplasm, providing dose-sparing effect. Depending on the PBC concentrations, the formulation can be a liquid micelle solution or a semi-solid thermo-reversible gel.

### Example 1

The proteins listed in Table 1 below were complexed with the PBC at ratios of 3:1, 9:1, and 30:1 w/w, PBC:protein.

TABLE 1

| | | Protein | Polymer |
|---|---|---|---|
| No. | Proteins (+PBC) | concentration (ug) | concentration (ug) |
| 1 | PBC | 0 | 15, 75, 150 |
| | (pentablock copolymer) | | |
| 2 | Insulin | 5 | 15, 75, 150 |
| 3 | Human serum Albumin | 5 | 15, 75, 150 |
| 4 | Semaglutide | 5 | 15, 75, 150 |
| 5 | HPV E7 | 5 | 15, 75, 150 |
| 6 | Myoglobin | 5 | 15, 75, 150 |
| 7 | Glucose oxidase | 5 | 15, 75, 150 |
| 8 | Amyloglucoseoxidase | 5 | 15, 75, 150 |
| 9 | Lysozyme | 5 | 15, 75, 150 |
| 10 | Interleukin 2 | 5 | 15, 75, 150 |

The complexation was conducted at room temperature by incubating the PBC with the protein for 10-20 minutes on a microcentrifuge. A formulation of PBC alone was prepared along with the protein and PBC complexes.

To prepare the formulations listed above, solutions of PBC and protein were first prepared. The PBC solution was prepared at a concentration of either 20 mg/ml or 40 mg/ml of PBC in phosphate buffered saline, pH 7.4 (PBS). The protein solutions were prepared at a concentration of 1 or 2 mg/ml in PBS. For complexation, the protein solution (1 or 2 mg/ml) was titrated with PBC solution (20 or 40 mg/ml. After addition of 1×PBS to give the appropriate concentrations, the mixture was centrifuged, then incubated to allow for complexation. All incubations were performed on a shaker at room temperature for 10-20 minutes.

### Analysis by Agarose Gel Electrophoresis

Formulation were prepared as described above and analyzed by agarose gel electrophoresis. A 2% agarose gel was prepared and 20 μl of each formulation was loaded at the center of the gel. The gel was run for 30 minutes at 75V. The gel was then visualized using 1-step blue protein stain™ The micellar complex remains close to the well but moves slightly out of well towards the negative electrode. The proteins tested move in the opposite direction towards the positive electrode.

### Results

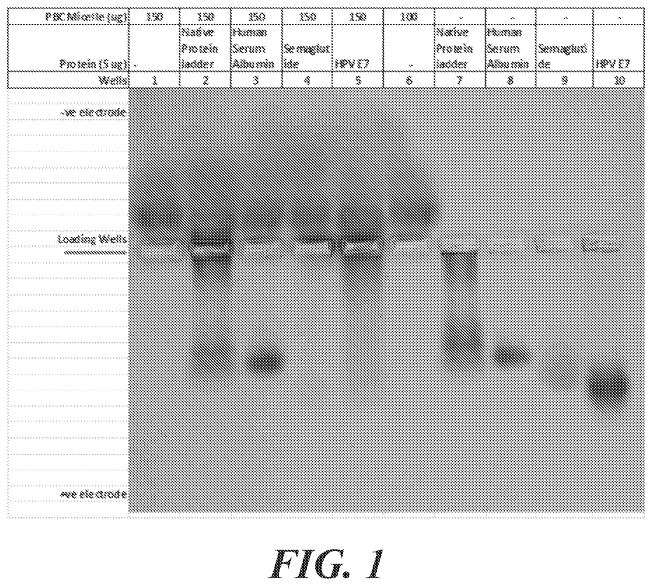

At the ratios studied, human serum albumin did not bind or complex with PBC. The other proteins complexed with PBC did. With reference to the results in FIG. 1, the PBC micellar complex was observed to move out of the well towards the negative electrode. The proteins, human serum albumin (HSA), semaglutide, and HPV E7 were observed to move towards the positive electrode. HSA was not observed to complex with the polymer as seen from the presence of the protein in well number 3. Semaglutide and HPV E7 were observed to complex with the polymer as seen from the absence of the proteins in wells 4 and 5 compared to wells 9 and 10.

### Example 2

In the following Example, the pentablock copolymer (“PBC”) micelles are made of a Pluronic F127 (poly(ethyleneoxide)-block-poly(propyleneoxide)-block poly(ethyleneoxide) (PEO-PPO-PEO) and pH-responsive cationic (poly(2-diethylaminoethyl methacrylate) (PDEAEM) as the end blocks. The amphiphilic Pluronic F127 blocks enhance cellular uptake, while the protonatable tertiary amine groups of PDEAEM facilitate endosomal escape and efficient payload release in the cytoplasm, providing dose-sparing effect. Depending on the PBC concentrations, the formulation can be a liquid micelle solution or a semi-solid thermo-reversible gel.

The proteins listed in Table 2 below were complexed with the PBC at weight ratios of PBC:protein as described below.

TABLE 2

| | w/w ratio | Protein |
|---|---|---|
| Protein | (PBC:protein) | (ug) |
| Semaglutide (no incubation) | 30:1 | 1.5 |
| Semaglutide | 30:1 | 1.5 |
| Semaglutide | 15:1 | 0.33 |
| Semaglutide | 15:1 | 1.5 |
| HPV E7 | 30:1 | 4.41 |
| HPV E7 | 15:1 | 4.5 |
| HPV E7 | 6.7:1 | 4.5 |
| Interleukin-2 | 15.3:1 | 4.5 |
| Interleukin-2 | 1:1 | 4.5 |
| Interferon alpha | 18:1 | 1.0 |
| Interferon gamma | 18:1 | 0.1 |
| Interferon gamma | 15:1 | 0.6 |

Unless otherwise indicated, the complexation was conducted at room temperature by incubating the PBC with the protein for 10-20 minutes on a microcentrifuge. A formulation of PBC alone was prepared along with the protein and PBC complexes.

### Analysis by Agarose Gel Electrophoresis

Formulation were prepared as described above and analyzed by agarose gel electrophoresis. A 2% agarose gel was prepared and 20 μl of each formulation was loaded at the center of the gel. The gel was run for 30 minutes at 75V. The gel was then visualized using 1-step blue protein stain™ The micellar complex remains close to the well but moves slightly out of well towards the negative electrode. The proteins tested move in the opposite direction towards the positive electrode.

### Results

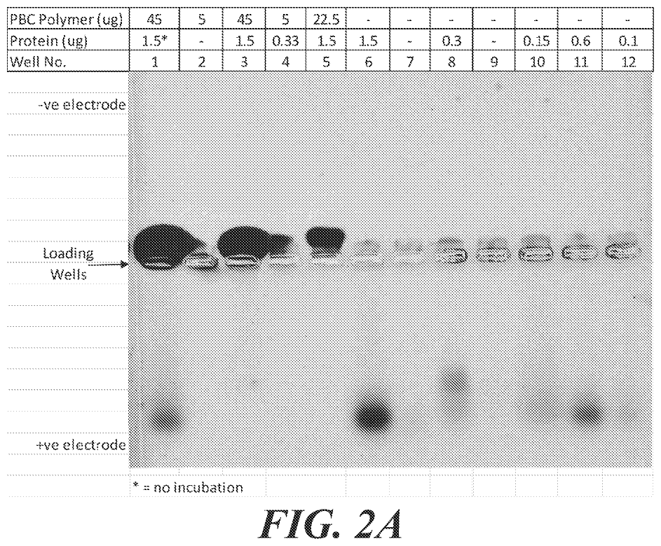

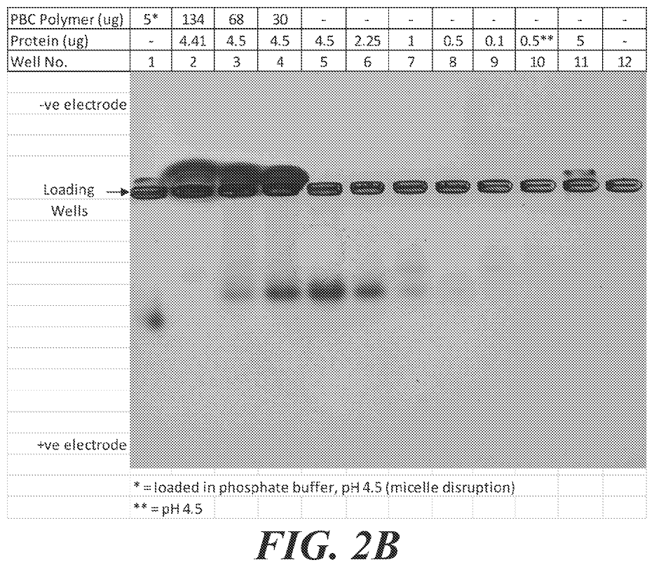

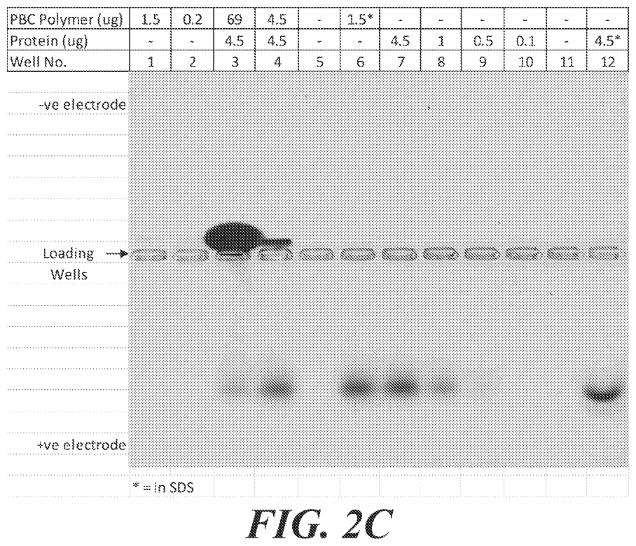

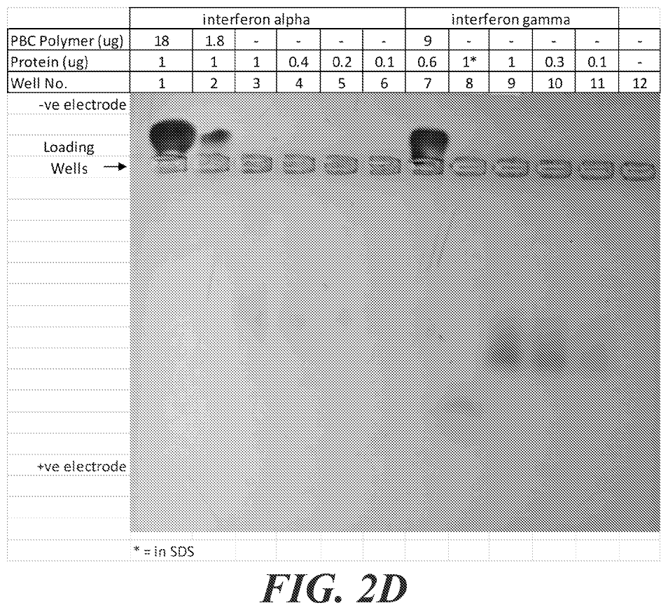

With reference to the results in FIGS. 2A-2D, the PBC micellar complex was observed to move out of the well towards the negative electrode. The proteins were observed to move towards the positive electrode. Semaglutide was observed to significantly complex with the polymer at polymer:protein ratios of 30:1 and 15:1, with or without incubation (FIG. 2A, see wells 1, 3, and 5 in comparison to well 6; well 4 in comparison to well 8). HPV E7 was observed to significantly complex with the polymer at polymer:protein ratios of 30:1 and 15:1 (FIG. 2B, see wells 2 and 3 in comparison to well 5); in addition, some complexation was observed at polymer:protein ratios of 6.7:1 (FIG. 2B, see well 4 in comparison to well 5). Interleukin-2 was observed to significantly complex with the polymer at polymer:protein ratios of 15:1 (FIG. 2C, see well 3 in comparison to well 7). Interferons alpha and gamma were observed to significantly complex with the polymer at polymer:protein ratios of 18:1 (interferon alpha, FIG. 2D, see well 1 in comparison to well 3) and 15:1, (interferon gamma, FIG. 2D, see well 7 in comparison to wells 9 and 10).

### Example 3

### Formulations of PBC with mRNA

In this example, cyanine-tagged EGFP mRNA was complexed with the PBC at N/P (N: positively charged amine groups in PBC; and P: negatively charged in mRNA) ratios of 10:1, 30:1, and 50:1. The complexation was conducted at room temperature by incubating the PBC with the mRNA for 30 minutes on a shaker. Formulations of PBC with mRNA (E, E1, E2, H, and H1), a formulation of free mRNA without any PBC (F), and a formulation of PBC without any mRNA (G) were prepared.

To prepare the formulations listed above, solutions of PBC and EGFP mRNA were first prepared. The PBC solution was prepared at a concentration of 20 mg/ml of PBC in RNAse-free 1× TBE buffer (Tris-borate-EDTA buffer). TBE is a buffer solution made up of Tris base, boric acid and EDTA. The EGFP mRNA was prepared at a concentration of 0.2 μg/μl by diluting 10 μl cyanine-tagged EGFP mRNA (1 μg/μl) with 45 μl 1× TBE buffer. The EGFP solution was mixed by vortexing and kept on ice. For complexation, cyanine-tagged EGFP mRNA (0.1 μg/μl) was titrated with PBC. 1× TBE was then added to the titration. After addition of 1× TBE, the mix was vortexed, then incubated to allow for complexation. All incubations were performed on a shaker at room temperature for 30 minutes.

Formulations of PBC with mRNA were prepared at N:P ratios of 10:1 (E), 30:1 (E1), and 50:1 (E2). For Formulation E, 2 μl of (20 μg/μl PBC) were mixed with 10 μl mRNA (0.2 μg/μl). Then, 8 μl of 1× TBE buffer were added before mixing by vortex. For preparing Formulation E1, 6 μl of (20 μg/μ1 PBC), 10 μl mRNA (0.2 μg/μl), and 4 μl of 1× TBE buffer were used. For preparing Formulation E2, 10 μl of (20 μg/μ1 PBC) and 10 μl mRNA (0.2 μg/μl) were used, without addition of 1× TBE.

The control mRNA formulation (F) was prepared by mixing 10 μl of 1× TBE buffer with 10 μl of EGFP mRNA (0.2 μg/μl). The control PBC formulation (G) was prepared by mixing 10 μl of 1× TBE buffer with 10 μl of PBC (20 μg/μl). As for formulations E, E1, and E2, the control formulations were incubated at room temperature for 30 minutes.

### Analysis by Gel Electrophoresis

Formulation E, E1, E2, F, and G were prepared as described above and analyzed by agarose gel electrophoresis. A 1% agarose gel was prepared and 20 μl of each formulation was loaded at the center of the gel. The gel was run for 30 minutes after addition of 5 μl of loading dye. The gel was then visualized on a fluorescence gel imager (LI-COR Odyssey).

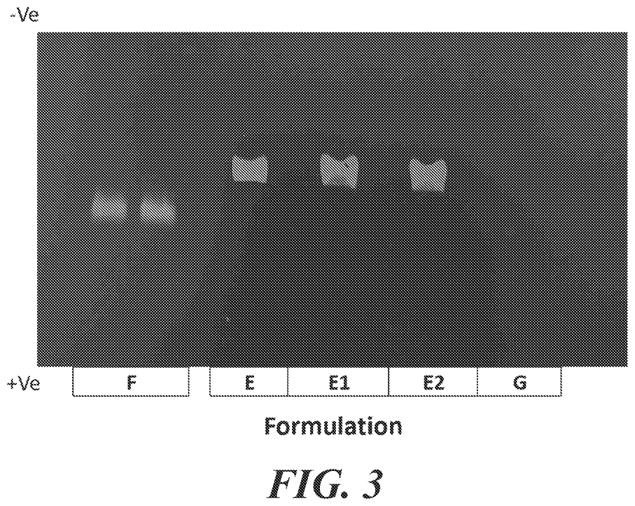

FIG. 3 shows a fluorescence image of the agarose gel result. The observed red fluorescence corresponds to signal from the cyanine-tagged EGFP. Cyanine is a red fluorescent tag. The gel image showed red fluorescence and captured the complexation of mRNA with PBC at different N:P ratios. In the gel. PBC-RNA complexes in Formulations E, E1, and E2 moved towards the negative electrode, while free mRNA in Formulation F moved towards the positive electrode. No fluorescence signal is detected in Formulation G.

In vivo Expression of EGFP Protein in Tumor

Two separate formulations (H and H1) of mRNA complexed with PBC were prepared and injected intra-tumorally in mice bearing xenograft tumors generated from a HCT116 colorectal cancer cell line.

Two formulations of PBC with mRNA were prepared for intra-tumoral injection. For preparation of formulation H, 20 mgs of PBC were dissolved in 0.5 ml of water to prepare a 40 mg/mL PBC solution. The PBC solution was vortexed until a homogenous solution was obtained. The pH of the PBC solution was not adjusted. 50 μl of PBC solution (40 mg/ml) were added 100 μl EGFP mRNA (1 μg/μl) and mixed by vortexing. For complexation, the preparation was incubated on a shaker for 30 mins at room temperature. Formulation H1 was prepared as Formulation H, except that PBC was dissolved in 10× TBE to prepare the PBC solution. Both Formulations H and H1 had an N:P ratio of 10:1.

### Example 4

### Formulations of PBC with mRNA

In this example, EPO mRNA encoding the human erythropoietin (EPO) protein, a hormone that controls erythropoiesis, or red blood cell production was complexed with the PBC at N/P (N: positively charged amine groups in PBC; and P: negatively charged in mRNA) ratios of 10:1, 30:1, and 50:1. The complexation was conducted at room temperature by incubating the PBC with the mRNA for 30 minutes on a shaker. Formulations of PBC with mRNA (E, E1, E2, H. and H1), a formulation of free mRNA without any PBC (F), and a formulation of PBC without any mRNA (G) were prepared. The EPO mRNA used in this case is a non-immunogenic EPO mRNA.

To prepare the formulations listed above, solutions of PBC and EPO mRNA were first prepared. The PBC solution was prepared at a concentration of 20 mg/ml of PBC in RNAse-free 1× TBE buffer (Tris-borate-EDTA buffer). TBE is a buffer solution made up of Tris base, boric acid and EDTA. The EPO mRNA was prepared at a concentration of 0.2 μg/μl by diluting 10 μl EPO mRNA (1 μg/μl) with 45 μl 1× TBE buffer. The EPO mRNA solution was mixed by vortexing at room temperature and kept on ice. For complexation, EPO mRNA (0.1 μg/μl) was titrated with PBC. 1× TBE was then added to the titration. After addition of 1× TBE, the mix was vortexed, then incubated to allow for complexation. All incubations were performed on a shaker at room temperature for 30 minutes.

Formulations of PBC with EPO mRNA were prepared at N:P ratios of 10:1 (A), 30:1 (A1), and 50:1 (A2). For Formulation A, 2 μl of (20 μg/μ1 PBC) were mixed with 10 μl mRNA (0.2 μg/μl). Then, 8 μl of 1× TBE buffer were added before mixing by vortex. For preparing Formulation E1, 6 μl of (20 μg/μl PBC), 10 μl mRNA (0.2 μg/μl), and 4 μl of 1× TBE buffer were used. For preparing Formulation E2, 10 μl of (20 μg/μ1 PBC) and 10 μl mRNA (0.2 μg/μl) were used, without addition of 1× TBE.

The control mRNA formulation (F) was prepared by mixing 10 μl of 1× TBE buffer with 10 μl of EPO mRNA (0.2 μg/μl). The control PBC formulation (G) was prepared by mixing 10 μl of 1× TBE buffer with 10 μl of PBC (20 μg/μl). As done for formulations A. A1, and A2, the control formulations were incubated at room temperature for 30 minutes.

Translation of EPO Protein Expression by PBC-EPO mRNA Formulations in Human Dendritic Cells

EPO-encoding mRNA formulations A, A1, A2, F, and G were prepared as described above. These were tested for their translational capacity in human dendritic cells (DCs). Cells were seeded into 96-well plates (1.5×10 cells/well) in 190 N1 of complete medium and transfected by adding 0.1 μg mRNA formulations in a 10-μl final volume. EPO levels were measured in the culture medium 24 hours post-transfection using an EPO ELISA kit.

Table 3 lists the results of the in vitro translation of EPO protein expression by the DC cells.

TABLE 3
Translational Capacity of PBC-EPO mRNA formulations in DCs

| PBC-EPO mRNA | EPO levels |
|---|---|
| formulation | (ng/ml) |
| A | 219 ± 18 |
| A1 | 248 ± 24 |
| A2 | 255 ± 28 |
| F | 0 |
| G | 0 |

As indicated by results (Table 3) of the in vitro translation by DCs, all the EPO-mRNA formulations at N/P ratios of 10:1, 30:1 and 50:1 translated EPO production. Levels of EPO were slightly lower DC cells transfected with PBC-EPO mRNA formulation A (N/P 10:1) but similar to formulations A1 and A2 (N/P: 30:1, 50:1 respectively) indicating stability and translation capacity of the EPO-mRNA formulation. Increasing levels of mRNA by proportionately increasing the levels of the PBC polymer would likely increase the levels of EPO translated by DC cells and in vivo.

### Embodiments

### Set 1

Embodiment 1. A polymeric micelle complex comprising:
   i) a plurality of block copolymers, wherein each block copolymer comprises at least a pentablock represented by formula (I) of

--[A]-[B]-[C]-[D]-[E]--,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer; and
ii) at least one therapeutic agent that is zwitterionic, wherein the therapeutic agent is hydrophobic or hydrophilic in aggregate,
   wherein different hydrophilic and/or hydrophobic regions and/or conformations of the therapeutic agent binds with complementary hydrophobic core and/or exterior hydrophilic layers of the polymeric micelle and/or at least a portion of the pendant moieties in the polymeric micelle to form a polymeric micellar complex.
Embodiment 2. The polymeric micelle complex of embodiment 1, wherein the blocks [A] and [E] each independently have a structure:

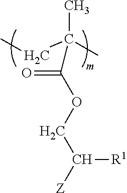

wherein
R¹ is selected from the group consisting of a hydrogen and a C₁₋₆ alkyl group;
Z is selected from the group consisting of NR²R³, P(OR⁴)₃, SR⁵,

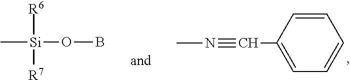

wherein R² and R³ are independently H, C₁₋₆ alkyl, or 1-mer to 28-mer oligonucleotide in which one or more of its natural phosphate backbone linkages are replaced with triazole linkages, or R² and R³ together with the nitrogen form a cyclic amine;
R⁴ is C₁₋₆ alkyl;
R⁵ is tri(C₁₋₆ alkyl) silyl; and
B is C₁₋₆alkyl;
and
in is an integer ranging from 1 to 5000.
Embodiment 3. The polymeric micelle complex of embodiment 2, wherein R¹ is H.
Embodiment 4. The polymeric micelle complex of embodiment 2 or 3, wherein Z is NR²R³ and at least one of R² and R³ is 1-mer to 28-mer oligonucleotide in which one or more of its natural phosphate backbone linkages are replaced with triazole linkages.
Embodiment 5. The polymeric micelle complex of embodiment 2 or 3, wherein Z is NR²R³ and R² and R³ together with the nitrogen form a cyclic amine.
Embodiment 6. The polymeric micelle complex of embodiment 5, wherein the cyclic amine is selected from the group consisting of pyrrolidine, piperidine, morpholine, and piperazine.
Embodiment 7. The polymeric micelle complex of embodiment 2 or 3, wherein Z is NR²R³ and R² and R³ are the same C₁₋₆ alkyl.
Embodiment 8. The polymeric micelle complex of embodiment 7, wherein R² and R³ are both ethyl.
Embodiment 9. The polymeric micelle complex of any one of embodiments 2-8, wherein
m is 13.
Embodiment 10. The polymeric micelle complex of any one of embodiments 1-9, wherein the blocks [A] and [E] are pH-responsive.
Embodiment 11. The polymeric micelle complex of any one of embodiments 1-10, wherein the pendant moieties of blocks [A] and [E] are cationic.
Embodiment 12. The polymeric micelle complex of any one of embodiments 1-11, wherein the alkylene oxide unit of the blocks [B] and [D] are unsubstituted and unbranched, and the alkylene oxide unit of the block [C] is substituted or branched.
Embodiment 13. The polymeric micelle complex of any one of embodiments 1-12, wherein the blocks [B] and [D] are the same

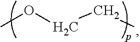

wherein p is an integer ranging from 30 to 20.000.
Embodiment 14. The polymeric micelle complex of embodiment 13, wherein the ratio of m:p is in the range of 0.1 to 1.
Embodiment 15. The polymeric micelle complex of embodiment 14, wherein m is about 13 and p is about 100.
Embodiment 16. The polymeric micelle complex of any one of embodiments 1-15, wherein the block [C] is

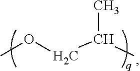

wherein q is an integer ranging from 1 to 20.000.
Embodiment 17. The polymeric micelle complex of embodiment 16, wherein the ratio of p:q is in the range of 10 to 1.
Embodiment 18. The polymeric micelle complex of embodiment 17, wherein p is about 100 and q is about 65.
Embodiment 19. The polymeric micelle complex of any one of embodiments 1-18, wherein the therapeutic agent is complexed to at least a portion of the pendant moieties via at least ionic interaction and or regional hydrophobicity or hydrophilicity within its structure and conformation.
Embodiment 20. The polymeric micelle complex of any one of embodiments 1-19, wherein at least one therapeutic agent is SARS-CoV-2 receptor-binding domain (RBD) protein, arginine deiminase, insulin, a Glucagon-like Peptide-1 (GLP-1) Receptor Agonist, Lepirudin, an Erythropoietin, Filgrastim, Glucagon, an Interferon, an Interleukin, a human papilloma virus (HPV) protein, or Desmopressin, or a derivative or metabolite of any of the foregoing.
Embodiment 21. The polymeric micelle complex of embodiment 20, wherein the Glucagon-like Peptide-1 (GLP-1) Receptor Agonist is lixisenatide, liraglutide, albiglutide, dulaglutide, semaglutide, or exenatide, or a derivative or metabolite of any of the foregoing.
Embodiment 22. The polymeric micelle complex of embodiment 20, wherein the Interferon is Interferon alfa-n3, Interferon beta, Interferon gamma, or Natural alpha interferon, or a derivative or metabolite of any of the foregoing.
Embodiment 23. The polymeric micelle complex of embodiment 20, wherein the Interleukin is Interleukin 2 (IL-2), IL-10, IL-12, transforming growth factor-β (TGF-β), IL-27, IL-35 or IL-37, or a derivative or metabolite of any of the foregoing.
Embodiment 24. The polymeric micelle complex of embodiment 20, wherein the human papilloma virus (HPV) protein is E1, E2, E4, E5, E6 or E1, or a derivative or metabolite of any of the foregoing.
Embodiment 25. The polymeric micelle complex of any one of embodiments 1-24, wherein at least one therapeutic agent is present at a molar ratio of zwitterionic agent:block copolymer ranging from about 0.01:1 to about 1:0.01 and/or in such an amount that the number of the functional group hearing a first charge in the block copolymer is at least 2 times or higher than the number of the functional group bearing the opposite charge in the first zwitterionic agent.
Embodiment 26. The polymeric micelle complex of any one of embodiments 1-25, wherein at least one therapeutic agent are two therapeutic agents.
Embodiment 27. The polymeric micelle complex of embodiment 26, wherein the two therapeutic agents are independently selected from the group consisting of: SARS-CoV-2 receptor-binding domain (RBD) protein, lepirudin, insulin, GLP-1 peptides, interferon, interleukins, and any derivatives or metabolites thereof.
Embodiment 28. The polymeric micelle complex of any one of embodiments 1-27, wherein the at least one therapeutic agent is present at a total molar ratio of zwitterionic agent:block copolymer ranging from about 0.01:1 to about 1:0.01 and/or in such an amount that the number of the functional group bearing a first charge in the block copolymer is at least 2 times or higher than the number of the functional group bearing the opposite charge in the first and second zwitterionic agent.
Embodiment 29. The polymeric micelle complex of any one of embodiments 1-28, further comprising a secondary agent.
Embodiment 30. The polymeric micelle complex of embodiment 29, wherein the secondary agent is a hydrophobic agent with a solubility greater than 10 μg/ml or greater than 10 ng/ml.
Embodiment 31. The polymeric micelle complex of embodiment 29 or 30, wherein the secondary agent is a therapeutic or diagnostic agent.
Embodiment 32. The polymeric micelle complex of embodiment 31, wherein the secondary agent is an imaging dye or a nucleic acid.
Embodiment 33. A pharmaceutical composition comprising the polymeric micelle complex of any one of embodiments 1-32, and a pharmaceutically acceptable carrier.
Embodiment 34. The pharmaceutical composition of embodiment 33, wherein the polymeric micelle complex formulated in a buffer in the pH range of 4.5-8.0 and/or other pharmaceutically acceptable solvents for parenteral administration.
Embodiment 35. A method for delivering at least one therapeutic agent that is zwitterionic to a human in need thereof, comprising administering to the human the polymeric micelle complex of any one of embodiments 1-32, or the pharmaceutical composition of embodiments 33 or 34.
Embodiment 36. A method for delivering at least one therapeutic agent that is zwitterionic to a cell interior of a subject in need thereof, comprising administering to the subject the polymeric micelle complex of any one of embodiments 1-32, or the pharmaceutical composition of embodiments 33 or 34, wherein at least a portion of the administered composition traverses the cell plasma membrane to deliver the at least one therapeutic agent to the cell interior.
Embodiment 37. A method for treating a disease in a human in need thereof, comprising administering to the human the polymeric micelle complex of any one of embodiments 1-32, or the pharmaceutical composition of embodiments 33 or 34.
Embodiment 38. The method of embodiment 37, wherein the disease is a cancer.
Embodiment 39. The method of embodiment 38, wherein the cancer is selected from the group consisting of colorectal cancer, gastrointestinal cancer, breast cancer, prostate cancer, and head & neck cancer.
Embodiment 40. The method of embodiment 37, wherein the disease is a microbial or viral infection.
Embodiment 41. The method of any one of embodiments 37-40, wherein at least one therapeutic agent is SARS-CoV-2 receptor-binding domain (RBD) protein, arginine deiminase, insulin, a Glucagon-like Peptide-1 (GLP-1) Receptor Agonist, Lepirudin, an Erythropoietin, Filgrastim, Glucagon, an Interferon, an Interleukin, a human papilloma virus (HPV) protein, or Desmopressin, a derivative or metabolite of any of the foregoing, or a combination of any of the foregoing.
Embodiment 42. The method of any one of embodiments 37-40, wherein at least one therapeutic agent is SARS-CoV-2 receptor-binding domain (RBD) protein, lepirudin, insulin, GLP-1 peptides, interferons, interleukins, a derivative or metabolite of any of the foregoing, or a combination of any of the foregoing.
Embodiment 43. The method of embodiment any one of embodiments 37-42, wherein at least a portion of the at least one therapeutic agent is delivered into the interior of a cell.
Embodiment 44. The method of any one of embodiments 37-43, wherein the polymeric micelle complex of any one of embodiments 1-32, or the pharmaceutical composition of embodiments 33 or 34 is administered by subcutaneously (SC), intravenously (IV) or intrathecally.
Embodiment 45. A kit comprising: the polymeric micelle complex of any one of embodiments 1-32, or the pharmaceutical composition of embodiments 33 or 34.
Embodiment 46. The kit of embodiment 45, further comprising a container and a label or package inserts) on or associated with the container.
Embodiment 47. Use of the polymeric micelle complex of any one of embodiments 1-32, or the pharmaceutical composition of embodiments 33 or 34 in therapy and/or diagnosis.
Embodiment 48. A method of preparing the polymeric micelle complex of any one of embodiments 1-32, or the pharmaceutical composition of embodiments 33 or 34.

### Set 2

Clause 1. A polymeric micelle complex comprising:
   i) a plurality of block copolymers, wherein each block copolymer comprises at least a pentablock represented by formula (I) of

--[A]-[B]-[C]-[D]-[E]--,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer; and
ii) mRNA coding for a peptide and/or protein, wherein mRNA binds with the pendant moieties in the polymeric micelle based on electrostatic interactions, and binds to the hydrophilic layers of the polymeric micelle to form a stable polymeric micellar complex.
Clause 2. The polymeric micelle complex of clause 1, wherein the blocks [A] and [E] each independently have a structure:

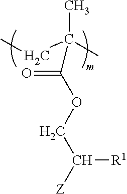

wherein
R¹ is selected from the group consisting of a hydrogen and a C₁₋₆ alkyl group;
Z is selected from the group consisting of NR²R³, P(OR⁴)₃, SR⁵,

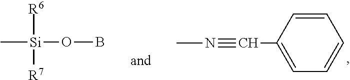

wherein R² and R³ are independently H, C₁₋₆ alkyl, or 1-mer to 28-mer oligonucleotide in which one or more of its natural phosphate backbone linkages are replaced with triazole linkages, or R² and R³ together with the nitrogen form a cyclic amine;
R⁴ is C₁₋₆ alkyl;
R⁵ is tri(C₁₋₆ alkyl) silyl; and
B is C₁₋₆alkyl;
and
m is an integer ranging from 1 to 5000.
Clause 3. The polymeric micelle complex of clause 2, wherein R¹ is H.
Clause 4. The polymeric micelle complex of clause 2 or 3, wherein Z is NR²R³ and at least one of R² and R³ is 1-mer to 28-mer oligonucleotide in which one or more of its natural phosphate backbone linkages are replaced with triazole linkages.
Clause 5. The polymeric micelle complex of clause 2 or 3, wherein Z is NR²R³ and R² and R³ together with the nitrogen form a cyclic amine.
Clause 6. The polymeric micelle complex of clause 5, wherein the cyclic amine is selected from the group consisting of pyrrolidine, piperidine, morpholine, and piperazine.
Clause 7. The polymeric micelle complex of clause 2 or 3, wherein Z is NR²R³ and R² and R³ are the same C₁₋₆ alkyl.
Clause 8. The polymeric micelle complex of clause 7, wherein R² and R³ are both ethyl.
Clause 9. The polymeric micelle complex of any one of clauses 2-8, wherein m is 13.
Clause 10. The polymeric micelle complex of any one of clauses 1-9, wherein the blocks [A] and [E] are pH-responsive.
Clause 11. The polymeric micelle complex of any one of clauses 1-10, wherein the pendant moieties of blocks [A] and [E] are cationic.
Clause 12. The polymeric micelle complex of any one of clauses 1-11, wherein the alkylene oxide unit of the blocks [B] and [D] are unsubstituted and unbranched, and the alkylene oxide unit of the block [C] is substituted or branched.
Clause 13. The polymeric micelle complex of any one of clauses 1-12, wherein the blocks [B] and [D] are the same

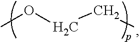

wherein p is an integer ranging from 30 to 20,000.
Clause 14. The polymeric micelle complex of clause 13, wherein the ratio of m:p is in the range of 0.1 to 1.
Clause 15. The polymeric micelle complex of clause 14, wherein m is about 13 and p is about 100.
Clause 16. The polymeric micelle complex of any one of clauses 1-15, wherein the block [C] is

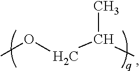

wherein q is an integer ranging from 1 to 20,000.
Clause 17. The polymeric micelle complex of clause 16, wherein the ratio of p:q is in the range of 10 to 1.
Clause 18. The polymeric micelle complex of clause 17, wherein p is about 100 and q is about 65.
Clause 19. The polymeric micelle complex of any one of clauses 1-18, wherein the first mRNA is complexed to at least a portion of the pendant moieties via at least ionic interaction.
Clause 20. The polymeric micelle complex of any one of clauses 1-19, wherein the mRNA codes for SARS-CoV-2 receptor-binding domain (RBD) protein, erythropoietin, a Glucagon-like peptide (GLP-1) Receptor Agonist, an Interferon, an Interleukin, or a human papilloma virus (HPV) protein, or a derivative and/or metabolite of any of the foregoing, or any combination thereof.
Clause 21. The polymeric micelle complex of clause 20, wherein the GLP-1 Receptor Agonist is lixisenatide, liraglutide, albiglutide, dulaglutide, semaglutide, or exenatide.
Clause 22. The polymeric micelle complex of clause 20, wherein the Interferon is Interferon alfa-n3, Interferon beta, Interferon gamma, or Natural alpha interferon.
Clause 23. The polymeric micelle complex of clause 20, wherein the Interleukin is Interleukin 2 (IL-2), IL-10, IL-12, transforming growth factor-β (TGF-β), IL-27, IL-35 or IL-37.
Clause 24. The polymeric micelle complex of clause 23, wherein the human papilloma virus (HPV) protein is E1, E2, E4, E5, E6 or E7.
Clause 25. The polymeric micelle complex of any one of clauses 1-24, wherein the mRNA is present at a molar ratio of mRNA:block copolymer ranging from about 0.01:1 to about 1:0.01 and/or in such an amount that the number of the functional group bearing a first charge in the block copolymer is at least 2 times or higher than the number of the negatively charged phosphate groups in the mRNA.
Clause 26. The polymeric micelle complex of any one of clauses 1-25, further comprising a second mRNA that is a different sequence, wherein the second mRNA complexes with at least a portion of the pendant moieties in the polymeric micelle.
Clause 27. The polymeric micelle complex of clause 26, wherein the second mRNA codes for SARS-CoV-2 receptor-binding domain (RBD) protein, erythropoietin, a (GLP-1) Receptor Agonist, an Interferon, an Interleukin, or a human papilloma virus (HPV) protein, or a derivative or metabolite of any of the foregoing, or any combination thereof.
Clause 28. The polymeric micelle complex of any one of clauses 1-27, wherein the mRNA is present at a total molar ratio of mRNA:block copolymer ranging from about 0.01:1 to about 1:0.01 and/or in such an amount that the number of the functional group bearing a first charge in the block copolymer is at least 2 times or higher than the number of negatively charged phosphate groups in the mRNAs.
Clause 29. The polymeric micelle complex of any one of clauses 1-28, further comprising a secondary agent.
Clause 30. The polymeric micelle complex of clause 29, wherein the secondary agent is a hydrophobic agent with a solubility greater than 10 μg/ml or greater than 10 ng/ml.
Clause 31. The polymeric micelle complex of clause 29 or 30, wherein the secondary agent is a therapeutic or diagnostic agent.
Clause 32. The polymeric micelle complex of clause 31, wherein the secondary agent is an imaging dye or a nucleic acid.
Clause 33. A pharmaceutical composition comprising the polymeric micelle complex of any one of clauses 1-32, and a pharmaceutically acceptable carrier.
Clause 34. The pharmaceutical composition of clause 33, wherein the polymeric micelle complex formulated in a buffer in the pH range of 4.5-8.0 and/or other pharmaceutically acceptable solvents for parenteral administration.
Clause 35. A method for delivering mRNA to a human in need thereof, comprising administering to the human the polymeric micelle complex of any one of clauses 1-32, or the pharmaceutical composition of clauses 33 or 34.
Clause 36. A method for delivering mRNA to a cell interior of a subject in need thereof, comprising administering to the subject the polymeric micelle complex of any one of clauses 1-32, or the pharmaceutical composition of clauses 33 or 34, wherein at least a portion of the administered composition traverses the cell plasma membrane to deliver the mRNA to the cell interior.
Clause 37. A method for treating a disease in a human in need thereof, comprising administering to the human the polymeric micelle complex of any one of clauses 1-32, or the pharmaceutical composition of clauses 33 or 34.
Clause 38. The method of clause 37, wherein the disease is a cancer.
Clause 39. The method of clause 38, wherein the cancer is selected from the group consisting of colorectal cancer, gastrointestinal cancer, breast cancer, prostate cancer, and head & neck cancer.
Clause 40. The method of clause 37, wherein the disease is a microbial or viral infection.
Clause 41. The method of any one of clauses 37-40, wherein the mRNA codes for: SARS-CoV-2 receptor-binding domain (RBD) protein, erythropoietin, a (GLP-1) Receptor Agonist, an Interferon, an Interleukin, a human papilloma virus (HPV) protein, or a derivative and/or metabolite thereof;
   or a combination of any of the foregoing mRNAs.
Clause 42. The method of any one of clauses 37-40, wherein the mRNAs codes for:
   SARS-CoV-2 receptor-binding domain (RBD), erythropoietin, interferons, interleukins, or a derivative and/or metabolite thereof;
   or any combinations thereof.
Clause 43. The method of any one of clauses 37-42, wherein at least a portion of mRNA is delivered into the interior of a cell, and wherein a protein encoded by said mRNA is expressed in a cell.
Clause 44. The method of any one of clauses 37-43, wherein the polymeric micelle complex of any one of clauses 1-32, or the pharmaceutical composition of clauses 33 or 34 is administered by subcutaneously (SC), orally (PO), intramuscularly (IM), intraperitoneally (IP), intravenously (IV) or intrathecally.
Clause 45. A kit comprising: the polymeric micelle complex of any one of clauses 1-32, or the pharmaceutical composition of clauses 33 or 34.
Clause 46. The kit of clause 45, further comprising a container and a label or package insert(s) on or associated with the container.
Clause 47. Use of the polymeric micelle complex of any one of clauses 1-32, or the pharmaceutical composition of clauses 33 or 34 in therapy and/or diagnosis.
Clause 48. A method of preparing the polymeric micelle complex of any one of clauses 1-32, or the pharmaceutical composition of clauses 33 or 34.

1. A polymeric micelle complex comprising:
i) a plurality of block copolymers, wherein each block copolymer comprises at least a pentablock represented by formula (I) of
--[A]-[B]-[C]-[D]-[E]--,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer, and an active agent selected from:
ii) at least one therapeutic agent that is zwitterionic, wherein the therapeutic agent is hydrophobic or hydrophilic in aggregate,
wherein different hydrophilic and/or hydrophobic regions and/or conformations of the therapeutic agent binds with complementary hydrophobic core and/or exterior hydrophilic layers of the polymeric micelle and/or at least a portion of the pendant moieties in the polymeric micelle to form a polymeric micellar complex; or
iii) mRNA coding for a peptide and/or protein,
wherein mRNA binds with the pendant moieties in the polymeric micelle based on electrostatic interactions, and binds to the hydrophilic layers of the polymeric micelle to form a stable polymeric micellar complex.

2. The polymeric micelle complex of claim 1, wherein the blocks [A] and [E] each independently have a structure: 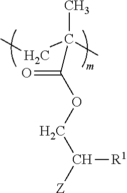
wherein
R¹ is selected from the group consisting of a hydrogen and a C₁₋₆ alkyl group;
Z is selected from the group consisting of NR²R³, P(OR⁴)₃, SR⁵, 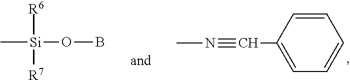
wherein R² and R³ are independently H, C₁₋₆ alkyl, or 1-mer to 28-mer oligonucleotide in which one or more of its natural phosphate backbone linkages are replaced with triazole linkages, or R² and R³ together with the nitrogen form a cyclic amine;
R⁴ is C₁₋₆ alkyl;
R⁵ is tri(C₁₋₆ alkyl) silyl; and
B is C₁₋₆ alkyl;
and
m is an integer ranging from 1 to 5000.

3. The polymeric micelle complex of claim 2, wherein R¹ is H.

4. The polymeric micelle complex of claim 2, wherein Z is NR²R³ and at least one of R² and R³ is 1-mer to 28-mer oligonucleotide in which one or more of its natural phosphate backbone linkages are replaced with triazole linkages.

5. The polymeric micelle complex of claim 2, wherein Z is NR²R³ and R² and R³ together with the nitrogen form a cyclic amine.

6. The polymeric micelle complex of claim 5, wherein the cyclic amine is selected from the group consisting of pyrrolidine, piperidine, morpholine, and piperazine.

7. The polymeric micelle complex of claim 2, wherein Z is NR²R³ and R² and R³ are the same C₁₋₆ alkyl.

8. The polymeric micelle complex of claim 7, wherein R² and R³ are both ethyl.

9. The polymeric micelle complex of claim 2, wherein m is 13.

10. The polymeric micelle complex of claim 1, wherein the blocks [A] and [E] are pH-responsive.

11. The polymeric micelle complex of claim 1, wherein the pendant moieties of blocks [A] and [E] are cationic.

12. The polymeric micelle complex of claim 1, wherein the alkylene oxide unit of the blocks [B] and [D] are unsubstituted and unbranched, and the alkylene oxide unit of the block [C] is substituted or branched.

13. The polymeric micelle complex of claim 1, wherein the blocks [B] and [D] are the same 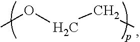
wherein p is an integer ranging from 30 to 20,000.

14. The polymeric micelle complex of 13, wherein the ratio of m:p is in the range of 0.1 to 1.

15. The polymeric micelle complex of 14, wherein m is about 13 and p is about 100.

16. The polymeric micelle complex of claim 1, wherein the block [C] is 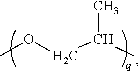
wherein q is an integer ranging from 1 to 20,000.

17. The polymeric micelle complex of 16, wherein the ratio of p:q is in the range of 10 to 1.

18. The polymeric micelle complex of 17, wherein p is about 100 and q is about 65.

19. The polymeric micelle complex of claim 1, wherein the polymeric micelle complex comprises:
i) a plurality of block copolymers, wherein each block copolymer comprises at least a pentablock represented by formula (I) of
--[A]-[B]-[C]-[D]-[E]--,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer, and
ii) at least one therapeutic agent that is zwitterionic, wherein the therapeutic agent is hydrophobic or hydrophilic in aggregate,
wherein different hydrophilic and/or hydrophobic regions and/or conformations of the therapeutic agent binds with complementary hydrophobic core and/or exterior hydrophilic layers of the polymeric micelle and/or at least a portion of the pendant moieties in the polymeric micelle to form a polymeric micellar complex.

20. The polymeric micelle complex of claim 19, wherein the therapeutic agent is complexed to at least a portion of the pendant moieties via at least ionic interaction and or regional hydrophobicity or hydrophilicity within its structure and conformation.

21. The polymeric micelle complex of claim 19, wherein at least one therapeutic agent is SARS-CoV-2 receptor-binding domain (RBD) protein, arginine deiminase, insulin, a Glucagon-like Peptide-1 (GLP-1) Receptor Agonist, Lepirudin, an Erythropoietin, Filgrastim, Glucagon, an Interferon, an Interleukin, a human papilloma virus (HPV) protein, or Desmopressin, or a derivative or metabolite of any of the foregoing.

22. The polymeric micelle complex of claim 21, wherein the Glucagon-like Peptide-1 (GLP-1) Receptor Agonist is lixisenatide, liraglutide, albiglutide, dulaglutide, semaglutide, or exenatide, or a derivative or metabolite of any of the foregoing.

23. The polymeric micelle complex of claim 21, wherein the Interferon is Interferon alfa-n3, Interferon beta, Interferon gamma, or Natural alpha interferon, or a derivative or metabolite of any of the foregoing.

24. The polymeric micelle complex of claim 21, wherein the Interleukin is Interleukin 2 (IL-2), IL-10, IL-12, transforming growth factor-β (TGF-β), IL-27, IL-35 or IL-37, or a derivative or metabolite of any of the foregoing.

25. The polymeric micelle complex of claim 21, wherein the human papilloma virus (HPV) protein is E1, E2, E4, E5, E6 or E7, or a derivative or metabolite of any of the foregoing.

26. The polymeric micelle complex of claim 19, wherein at least one therapeutic agent is present at a molar ratio of zwitterionic agent:block copolymer ranging from about 0.01:1 to about 1:0.01 and/or in such an amount that the number of the functional group bearing a first charge in the block copolymer is at least 2 times or higher than the number of the functional group bearing the opposite charge in the first zwitterionic agent.

27. The polymeric micelle complex of claim 19, wherein at least one therapeutic agent are two therapeutic agents.

28. The polymeric micelle complex of claim 27, wherein the two therapeutic agents are independently selected from the group consisting of: SARS-CoV-2 receptor-binding domain (RBD) protein, lepirudin, insulin, GLP-1 peptides, interferons, interleukins, and any derivatives or metabolites thereof.

29. The polymeric micelle complex of claim 19, wherein the at least one therapeutic agent is present at a total molar ratio of zwitterionic agent:block copolymer ranging from about 0.01:1 to about 1:0.01 and/or in such an amount that the number of the functional group bearing a first charge in the block copolymer is at least 2 times or higher than the number of the functional group bearing the opposite charge in the first and second zwitterionic agent.

30. The polymeric micelle complex of claim 1, wherein the polymeric micelle complex comprises:
i) a plurality of block copolymers, wherein each block copolymer comprises at least a pentablock represented by formula (I) of
--[A]-[B]-[C]-[D]-[E]--,
wherein the repeating units of blocks [A] and [E] each independently comprise a pendant moiety carrying a first charge, and
wherein blocks [B], [C] and [D] are independently poly(alkylene oxide);
wherein the plurality of pentablock copolymers are arranged into a polymeric micelle with an interior hydrophobic core and an exterior hydrophilic layer, and
iii) mRNA coding for a peptide and/or protein, wherein mRNA binds with the pendant moieties in the polymeric micelle based on electrostatic interactions, and binds to the hydrophilic layers of the polymeric micelle to form a stable polymeric micellar complex.

31. The polymeric micelle complex of claim 30, wherein the first mRNA is complexed to at least a portion of the pendant moieties via at least ionic interaction.

32. The polymeric micelle complex of claim 30, wherein the mRNA codes for SARS-CoV-2 receptor-binding domain (RBD) protein, erythropoietin, a Glucagon-like peptide (GLP-1) Receptor Agonist, an Interferon, an Interleukin, or a human papilloma virus (HPV) protein, or a derivative and/or metabolite of any of the foregoing, or any combination thereof.

33. The polymeric micelle complex of claim 32, wherein the GLP-1 Receptor Agonist is lixisenatide, liraglutide, albiglutide, dulaglutide, semaglutide, or exenatide.

34. The polymeric micelle complex of claim 32, wherein the Interferon is Interferon alfa-n3, Interferon beta, Interferon gamma, or Natural alpha interferon.

35. The polymeric micelle complex of claim 32, wherein the Interleukin is Interleukin 2 (IL-2), IL-10, IL-12, transforming growth factor-β (TGF-β), IL-27, IL-35 or IL-37.

36. The polymeric micelle complex of claim 32, wherein the human papilloma virus (HPV) protein is E1, E2, E4, E5, E6 or E7.

37. The polymeric micelle complex of claim 30, wherein the mRNA is present at a molar ratio of mRNA:block copolymer ranging from about 0.01:1 to about 1:0.01 and/or in such an amount that the number of the functional group bearing a first charge in the block copolymer is at least 2 times or higher than the number of the negatively charged phosphate groups in the mRNA.

38. The polymeric micelle complex of claim 30, further comprising a second mRNA that is a different sequence, wherein the second mRNA complexes with at least a portion of the pendant moieties in the polymeric micelle.

39. The polymeric micelle complex of claim 38, wherein the second mRNA codes for SARS-CoV-2 receptor-binding domain (RBD) protein, erythropoietin, a (GLP-1) Receptor Agonist, an Interferon, an Interleukin, or a human papilloma virus (HPV) protein, or a derivative or metabolite of any of the foregoing, or any combination thereof.

40. The polymeric micelle complex of claim 30, wherein the mRNA is present at a total molar ratio of mRNA:block copolymer ranging from about 0.01:1 to about 1:0.01 and/or in such an amount that the number of the functional group bearing a first charge in the block copolymer is at least 2 times or higher than the number of negatively charged phosphate groups in the mRNAs.

41. The polymeric micelle complex of claim 1, further comprising a secondary agent.

42. The polymeric micelle complex of claim 41, wherein the secondary agent is a hydrophobic agent with a solubility greater than 10 μg/ml or greater than 10 ng/ml.

43. The polymeric micelle complex of claim 41, wherein the secondary agent is a therapeutic or diagnostic agent.

44. The polymeric micelle complex of claim 42, wherein the secondary agent is an imaging dye or a nucleic acid.

45. A pharmaceutical composition comprising the polymeric micelle complex of claim 1, and a pharmaceutically acceptable carrier.

46. The pharmaceutical composition of claim 45, wherein the polymeric micelle complex formulated in a buffer in the pH range of 4.5-8.0 and/or other pharmaceutically acceptable solvents for parenteral administration.

47. A method for delivering mRNA or at least one therapeutic agent that is zwitterionic to a human in need thereof, comprising administering to the human the polymeric micelle complex of claim 1.

48. A method for delivering mRNA or at least one therapeutic agent that is zwitterionic to a cell interior of a subject in need thereof, comprising administering to the subject the polymeric micelle complex of claim 1, wherein at least a portion of the administered composition traverses the cell plasma membrane to deliver the at least one therapeutic agent to the cell interior.

49. A method for treating a disease in a human in need thereof, comprising administering to the human the polymeric micelle complex of claim 1.

50. The method of claim 47, wherein the disease is a cancer.

51. (canceled)

52. The method of claim 49, wherein the disease is a microbial or viral infection.

53. (canceled)

54. (canceled)

55. The method of claim 49, wherein at least a portion of the mRNA or the at least one therapeutic agent is delivered into the interior of a cell.

56. The method of claim 55, wherein a protein encoded by said mRNA is expressed in the cell.

57. The method of claim 49, wherein the polymeric micelle complex is administered by subcutaneously (SC), orally (PO), intramuscularly (IM), intraperitoneally (IP), intravenously (IV) or intrathecally.

58-60. (canceled)

61. A method of preparing the polymeric micelle complex of claim 1.
